(19) United States
(12) Patent Application Publication
(10) Pub. No.: US 2024/0148541 A1
(43) Pub. Date: May 9, 2024

(54) SYSTEM AND DEVICE FOR URINATION

(71) Applicant: Ravi Ganesh ATHALYE, Maharashtra (IN)

(72) Inventor: Ravi Ganesh ATHALYE, Maharashtra (IN)

(21) Appl. No.: 18/549,700

(22) PCT Filed: Mar. 3, 2022
(86) PCT No.: PCT/IN2022/050186

§ 371 Date: Sep. 8, 2023

(30) Foreign Application Priority Data
Mar. 11, 2021 (IN) 202021039261

(51) Int. Cl. A61F 5/451; A61F 5/44; A61G 9/00

(52) U.S. Cl. CPC A61F 5/451; A61F 5/4404; A61G 9/006

(57) ABSTRACT

A system and device are provided for self-help urination for bed ridden users as well as females to enable urinating standing up. The system enables auto-monitoring of urination of in-cognitive bedridden patients or cognitive patients and evacuating the urine to a storage canister. Currently, such patients are invasively inserted catheters to evacuate urine with subsequent vulnerability to infections and expensive post-operation processes. The device is provided with the capability to capture a flowing stream of urine. In a flowing stream, any escape of fluid from the vacuum zone is manifest as leakage which is not acceptable. The invention provides genital device customized to males and females, to safely vacuum the urine by placing it on the genitals without discomfort to the patient. The process of introducing air during suction also dries the genitals thus significantly reducing the risks of infection.

### FIELD OF THE INVENTION

The present invention relates to urination devices and more particularly to a system and device for self-help urination in cognitive and in-cognitive conditions.

### BACKGROUND OF THE INVENTION

Patients who are immobile due to any number of reasons like surgery or sickness, the use of a bedpan is unavoidable. It involves lifting the user over the bedpan. The nature of immobility, weight of the person, and connected auxiliaries like saline tubes, make it challenging and at times, unsafe to execute such procedure. Further, the deficiency after use cleanup of the patient can cause infection leading to expensive medical intervention.

Senior males often develop frequent nocturnal urination. The trips to the restroom with impaired vision, balance, strength makes urination a hazardous undertaking. There are portable carry-on urine collection units that cater to customers who are active and have frequent urination. Though the devices are portable they are not suited for bedridden patients since they depend on gravity to transport the urine to the container. Gravity imposes constraints in the positioning of the container which must be placed below the level of the patient. These devices are incapable of leakage-free function when the patient is in a lying-down position. The current state of art shows the method has many incidences of leakage. Further, the devices are not self-cleaning.

Another known inconvenience is that females have to urinate sitting down on a commode or in absence of which, squatting, unlike males who can urinate standing up. There exist urination devices that enable females to urinate standing up. However, the devices are like funnel cups and have significant issues of embarrassing leakage that makes the task of urination cumbersome.

Therefore, there is a need for a system and device that facilitates self-help urination in cognitive and in-cognitive conditions without leakage as well as enable females to urinate standing up without embarrassing leakage.

### SUMMARY OF THE INVENTION

The present invention provides a system for urination for bedridden users both cognitive and in-cognitive, and a system for urination in portable device to enable females to urinate standing up. A version for female toilets with disposable urination devices is also an embodiment included in this invention.

The present invention provides a urination device that is configured to be uniformly implemented between a system for bed ridden users and in devices for females to urinate standing up. The urination device creates an aerosol mixture of urine through introduction of directed streams of air all along the interface of the device with the genitals, for both males and females when connected to a vacuum pump. The device's low volume profile ensures a high velocity of inrush air necessary for urine to be picked up and transported to the vacuum port in the device. Since the design requires air introduction to function, urine leakage due to lack of sealing of the device with the genitals is nearly eliminated.

The urination device forms the vital link connecting the human genitals to the vacuum pump. The Urination device is uniquely designed to capture the urine without discomfort to the human through the suction effected by vacuum pump. The urine is converted to an aerosol vapor of urine and air and the mixture propagated in the direction of the vacuum pressure from the vacuum pump. The vacuum pump is either manually turned ON/OFF or through sensors driven by a control logic. The urination device has design features to progressively transport a body of urine, both stagnant and streaming with minimally imparting any force on the genitals. The urination device needs to be sanitized between use to eliminate any infections.

In one aspect, the present invention provides a system for urination device. The system comprises an enclosure having a sanitization fluid container and an overflow container placed therein. A sanitization tray is placed in the enclosure. The sanitization tray is configured to facilitate placement of an urination device therein. A control circuit is provided for filling and vacuuming the sanitization tray for sanitizing the urination device. A vacuum pump is connected with the control circuit for vacuuming the urine output of the user through the urination device. A thumb switch is configured on a handle of the urination device. The thumb switch is configured to turn ON/OFF the vacuum pump. A timer circuit is configured to receive the thumb switch input to turn ON the vacuum pump for pumping sanitization fluid into a sanitization tray to a predetermined level. The sanitization tray has a drain port and an overflow port. The thumb switch is connected to a low voltage DC source that powers a coil of a relay.

In an embodiment, the system for bed ridden users uses a sanitization rinsing tray which conducts rinsing by sucking sanitization fluid through the urination device and the connecting tube by introducing a measured volume of sanitization fluid into the rinsing tray which is the parking spot for the urination device between use thereof.

The controls for sensing the presence of the urination device in the tray are through a combination of sensors and control box that comprehensively provides logic for turning the vacuum pump On/Off both during urine evacuation and the sanitization of the urination device.

The urination device controls vary between the type of user. Holding methods for the urination device, the type of suction pump, the operating switch and power source have customized shape and locations based on the type of the user.

For bed ridden users who are cognitive a thumb switch is configured on a handle of the urination device. The thumb switch is part of the overall logic to turn On/Off the vacuum pump. A control box, sanitization fluid container, a pump to transfer fluid from the sanitization container to the tray, an overflow container for the tray and the vacuum pump that vacuums both the urine, and the rinsing fluid is part of the system. An appropriate logic for evacuating urine during urination and the evacuation of the sanitization fluid during rinsing is all enabled through sensors, timer and switches.

The rinsing tray is provided with an inlet port for pumping sanitization fluid into the rinsing tray. The control box with timer controls and sensors enables the rinsing function. The control box has a timer circuit configured to receive the thumb switch input to turn ON the vacuum pump for pumping sanitization fluid into a sanitization tray to a predetermined level. The sanitization tray has a drain port and an overflow port. The thumb switch is connected to a low voltage DC source that powers a coil of a relay.

In another aspect, the present invention provides a urination device that comprises a shallow body with a plurality of arches on an outer periphery thereof. A plurality of legs is configured at the periphery radiating beyond the arches thereby forming a plurality of open channels for extending a suction zone. A plurality of opposing high velocity air channels is configured on an inner surface of the body in a predefined pattern that act as an air ports for inrush air. The channel walls ensure an air buffer between the organ on which the cup is placed and the suction end of the suction spout. An outlet is positioned on the body forming a vacuum tube connector spout. A tube is connected to the vacuum tube connector spout at a first end of the tube and a second end of the tube connected to a wet vacuum pump device. The body of the device includes a mesh unit. The mesh unit comprises a pair of conducting mesh separated by a fixed distance arranged to cover the open surface of the device that are part of twin elements of sensors for detecting the presence of urine when connected to a commensurate circuit, the pair of mesh being connected to conductors that are inserted through the cup surface to be available for connecting the sensors to a control circuit. The inner surface of the body of the device includes at least one sensor for detection of the urine received therein. An outer surface of the device includes a plurality of extension loops that receive a strap for placing the device on the body of the user.

The urination device comprises a shallow cup shaped body with a plurality of arches on the outer periphery thereof. A plurality of legs is configured at the periphery radiating beyond the arches thereby forming a plurality of open channels for extending the suction zone. A plurality of opposing high velocity air channels is configured on an inner surface of the body in a predefined pattern that act as an air ports for inrush air. The channel walls have sufficient height to ensure an air buffer between the genital on which the cup is placed and the suction end of the suction spout. An outlet is positioned on the body of the urination device, forming a vacuum tube connector spout. A tube is connected to the vacuum tube connector spout at a first end of the tube and a second end of the tube connected to a wet vacuum pump device.

The embodiment for in-cognitive bed ridden users, includes a urine sensor in the forms of parallel conducting mesh nets separated by a predetermined distance. The mesh unit when wetted with urine establishes a conduction bridge between the two. The pair are connected to the control box to signal the presence or absence of urine. The control box thus turns On/Off the vacuum pump based on the presence or absence of urine in the urination device.

The inner surface of the body of the device includes at least one sensor for detection of the urine received therein. An outer surface of the device includes a plurality of extension loops that receive a strap for placing and affixing the urination device to the body of the user.

It must be noted that the vacuum force for bed ridden users evacuates urine against gravity and therefore needs a sufficiently powerful motor propeller to create the necessary vacuum.

A portable embodiment of the present invention includes an urination device described above in the form of shallow cup shaped body with a plurality of arches on an outer periphery thereof. It is to be noted here that in this one embodiment the urination device is disposable and can be press fitted in a vacuum tube assembly fixed to the wall or any such structure thereof. A plurality of legs is configured at the periphery radiating out of the ends of arches thereby forming a plurality of open channels for extending a suction zone. A plurality of opposing high velocity air channels is configured on an inner surface of the body in a predefined pattern that act as an air ports for inrush air. The channel walls ensure an air buffer between the genital on which the cup is placed and the suction end of the suction spout. A plurality of high velocity air channels is configured on an inner surface of the body in a predefined pattern. The urination device is a convex nearly flat surface that covers the genitals. The minimal volume ensures the air introduced through ports has a high velocity. An outlet in the form of a spout is positioned at a predefined portion of the body. The spout is sufficiently long to have space for placement of a compact motor propeller combination to create the necessary vacuum for the suction head. A novel system of a nebulizer sieve, a diverter cone and coaxial shells enable the urine to be channeled through the spout past the motor without wetting it. The spout is tapered at its tip to enable recombination of the urine aerosol vapor into a stream. It is to be noted since this version is used by females for urination standing up, the spout, akin to a male phallus, and is naturally positioned below the level of the urination device. This stance ensures that gravity assists the urine vapor to flow downwards thereby allowing the motor propeller to be compact and provide just sufficient vacuum to prevent leakage. The vacuum force required to transport urine is considerably reduced. The power to the motor is through wires connecting the switch to the motor. The portable personal device is made of disposable materials such as PET, Plant Fiber and the like.

The vacuum pump assembly comprises in the cascaded sequence. A tube includes a suction end and an exhaust end. The tube has a first section and a second section. The first section includes a switch embedded in the suction end of the tube one, a mesh diaphragm nebulizer, a cone separator, and a propeller shaped to provide a positive pressure along a limited zone around the axis and vacuum i.e. a negative pressure along the major portion away from the pressure zone extending to the tip of the propeller. The second section includes an inner shell to contain the motor. The motor is powered through a wire leading out of the co-axial shells to a power source through a plug. An outer shell is provided as a transport corridor between the inner shell and the outer shell and to integrate the vapor into a coalesced unified stream by tapering the shell. The vacuum pump assembly includes a wire that bridges the switch to the motor and an outlet defined at the end portion of the second section. The cone separator splits the urine droplets and channelizes it around the motor. The motor is positioned in a housing that forms a cavity between the motor's outer shell and the housing inner shell surface. The cavity defines an annular ring. The device is made of disposable materials such as PET, Plant Fiber and the like.

The version that is affixed in the toilet is similar to the portable unit described above except the urination device is detachably fixed to the inlet of the suction tube and is made of a disposable material. The body of the urination device and its outlet which is an integral part of the urination device is made of disposable material; and the vacuum tube assembly is made of PET or other material that is permanently affixed to the wall of the toilet. A limit switch is defined at the inlet portion of the first section of the vacuum tube assembly. The vacuum is turned On and Off upon insertion and removal.

### BRIEF DESCRIPTION OF THE DRAWING

FIG. 1 shows a system for self-help urination in accordance with an embodiment of the present invention;

FIG. 2 shows a male urination device positioned within a tray of the system of FIG. 1;

FIGS. 3A-3C show structural details of the male urination device adapted for bed-ridden male user in accordance with an embodiment of the present invention;

FIGS. 4A-4B show structural details of a female urination device adapted for bed-ridden female user in accordance with an embodiment of the present invention;

FIGS. 5A to 6E show a female urination device adapted to be utilized for bed-ridden female user in-cognitive condition in accordance with an alternative embodiment of the present invention;

FIGS. 7A-7C show a female urination device adapted for female user in a cognitive condition for urinating in a standing position in accordance with an alternative embodiment of the present invention; and

FIGS. 8A-8B show a female urination device of FIGS. 7A-7C configured to be utilized in public urinals in alternative embodiment of the present invention.

### DETAILED DESCRIPTION OF THE INVENTION

Although specific terms are used in the following description for sake of clarity, these terms are intended to refer only to particular structure of the invention selected for illustration in the drawings and are not intended to define or limit the scope of the invention.

In general aspect, the present invention relates to a system and device for self-help urination. The system enables auto-monitoring of urination of in-cognitive bedridden patients or cognitive patients and evacuating the urine to a storage canister. Currently, such patients are invasively inserted catheters to evacuate urine with subsequent vulnerability to infections and expensive post-operation processes. The device is provided with the capability to capture a flowing stream of urine. In a flowing stream, any escape of fluid from the vacuum zone is manifest as leakage which is not acceptable. The invention provides genital device customized to males and females, to safely vacuum the urine by placing it on the genitals without discomfort to the patient. The process of introducing air during suction also dries the genitals thus significantly reducing the risks of infection. The current invention eliminates leakage by implementing vacuum thus eliminating the need to skillfully hold a funnel around the organ to establish a leak free seal. The present invention provides a genital device in the form of a cup, customized to males and females, to safely vacuum the urine by placing it on the genitals without discomfort to the patient. The cup has a spout to which is connected a vacuum pump and inlet arches on the cup edge to allow air to be sucked through them. The process of introducing air during suction also dries the genitals thus significantly reducing the risks of infection.

The present invention is illustrated with reference to the accompanying drawings, throughout which reference numbers indicate corresponding parts in the various figures.

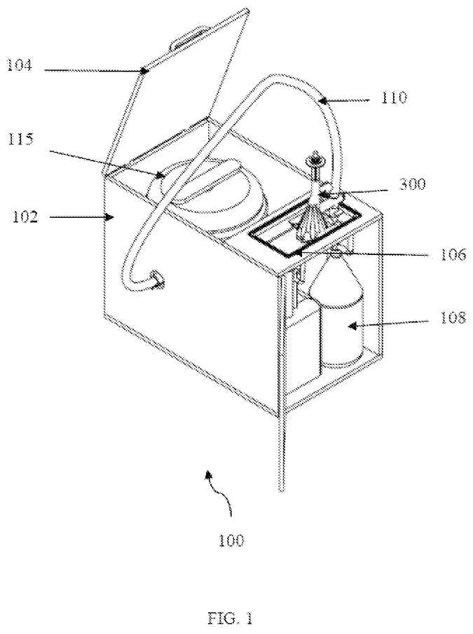

Referring to FIG. 1, a system (100) for urination in accordance with an embodiment of the present invention is shown. The system (100) includes an enclosure (102) having a lid (104) with a latch (not shown). In an embodiment, the latch is a magnetic latch. The lid (104) includes a sanitization tray cradle (106) cut out. The enclosure (102) facilitates placement of a sanitization fluid container (not shown), a vacuum pump (115) and an overflow container (not shown) therein that may be accessed by a side opening door with a magnetic latch. The enclosure (102) includes a removable partition on which a plurality of controls is mounted. The enclosure (102) is provided with cutouts on one of the sides to facilitate connection of electrical wires and tubing inside the enclosure (102) and exhaust port. The wire connects a control box to a power source.

In accordance with an embodiment, a sanitization tray (107) is placed in the tray cradle (106) and used for sanitization of an urination device (hereinafter referred as (device (300,400)). The process of sanitization is carried out by the sequential working of a control circuit. The tray (107) is filled with a sanitization fluid. The sanitization fluid is taken in the tray (107) from the sanitization fluid container. The tray (107) is filled simultaneously with the operation of a thumb switch (410)) on the device handle. In the next step, the sanitization fluid is returned to the tray (107) by vacuum pump mechanism (115).

In accordance with the present invention, a vacuum pump is utilized to supply vacuum to the system (100). In an embodiment, the vacuum pump (115) is a wet pick-up vacuum cleaner. However, various types of other vacuum pumps known in the art may be utilized in conjunction with the present invention. The vacuum pump has a vacuum port and an exhaust port. The vacuum port is connected to a light flexible vacuum tube that is connected via a swivel joint to the spout of the urination device (300, 400). The exhaust port is provided with a flap that is pushed open by exhaust air and closes when the air stops. The flap closure is to prevent any odor from the stored urine from escaping.

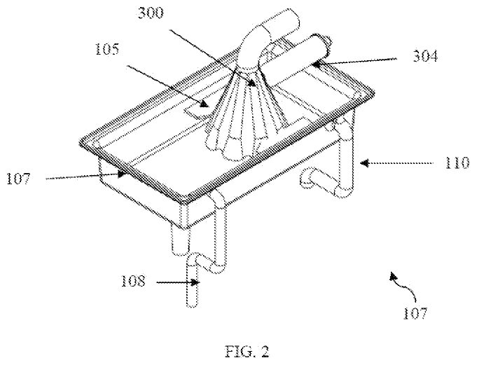

Referring to FIG. 2, a perspective view of the tray (107) having the urination device (300, 400) placed therein is shown. The tray (107) acts as a receptacle for the urination device (300, 400). The urination device (300, 400) is parked in the tray (107) such that open face of the urination device faces bottom of the tray (107). This is achieved by at least one overhang (105) provided in the tray (107) wherein the device (300,400) can be slid in. The overhang (105) captures the tube spout in a cradle (106) while legs of the device (300, 400) are pinned on two sides under the side wall overhang of the tray (107). In an embodiment, three overhung point supports are provided at predefined locations of the tray cradle (106) to enable the device (300, 400) to be erect in the tray (107).

In accordance with an embodiment, the tray (107) includes an overflow port (108) and a drain port (110). The overflow port (108) prevents any excess spillover of the sanitization fluid by routing it to the overflow container. The drain port (110) drips out any sanitization fluid missed by vacuum evacuation. This ensures that the device (200) is dry and ready for the next use. The tray (107) is tapered for easy insertion into the lid (104) opening of the enclosure (102). The tray (107) has bezels to enable it to hang in the opening of the enclosure (102). The tray (107) and the enclosure (102) in combination allows the tray (107) to be immobile once placed into the opening of the enclosure top.

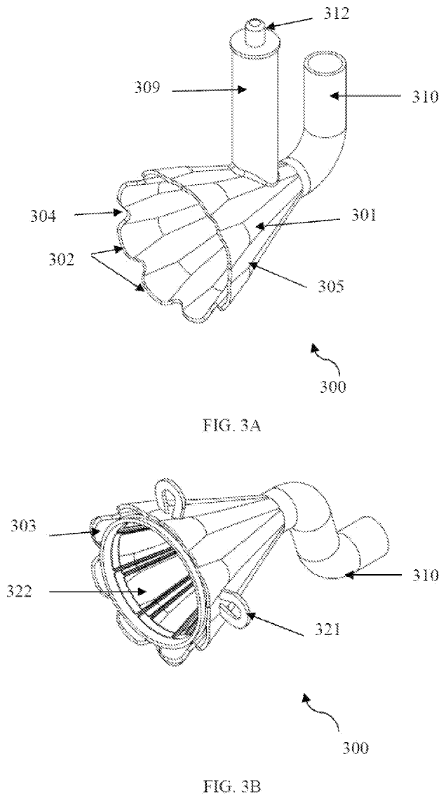

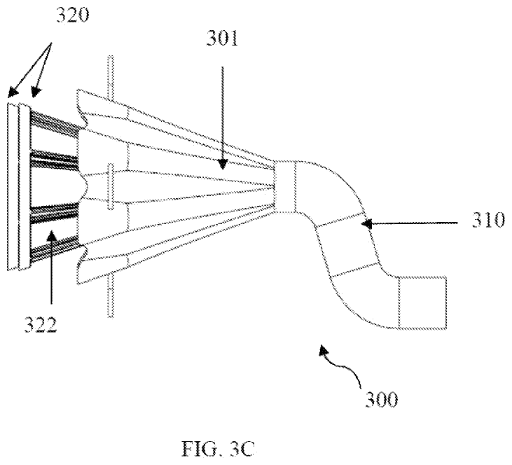

Referring to FIG. 3A-3C, a urination device (300) for male user (hereinafter referred as (male device (300)) in accordance with an embodiment of the present invention is shown. In an embodiment, the male device (300) has a conical body (301). The base of the cone acts as an entry face. The face has an undulated contour (304) for enabling air inrush in the male device (300) while in use. It is to be noted here, that conical body (301) has a plurality of arches (305) configured thereon. A plurality of legs (302) are configured at the periphery radiating beyond the arches (305) thereby forming a plurality of open channels for extending a suction zone. It is understood that shape and configuration of the conical shape body (301) may vary in other alternative embodiments of the present invention.

The conical body (301) has a top portion provided with an opening that acts as a vacuum tube connector spout (308). The vacuum tube connector spout (308) includes a tube (310) attached thereto at first end of the tube (310). The second end of the tube (310) is connected to a wet vacuum pump device (115). The perimeter and surface is provided with the undulated contour and the length long enough for the organ to be sufficient distance from the suction end. The undulated contour (304) ensures unhindered air streams all around the organ thereby ensuring the transport of urine. The conical shape of the device body (301) progressively promotes acceleration of the air velocity along with the urine towards the suction end. Air vents in the form of high velocity air channels (303) configured on inner surface of the device (300) to ensure inrush air to propel the urine to the suction port.

The shape and configuration of the male device (300) facilitates to capture the male genitalia while maintaining sufficient uninterrupted air flow around it for vacuum and transport of urine to an enclosure. The considerations for proportion and shape of the components of the conical body (301) are to guard the organ from being stressed due to vacuum and prevent it from being sucked into the vacuum port.

In another embodiment, the device (300) has two conductors (320) inserted in the suction exit end of the device (300) such that any spontaneous urination causes the urine to trickle down to the suction end making a conducting bridge contact between the conductors (320) due to wetting. At least one strap loop (321) is provided on the outer surface of the body (301). A mesh unit between the conductors (320) forms a conducting bridge due to wetting. The sensors and the conducting bridge activate the vacuum pump (115) to be switched ON. The conductors (320) are connected to an operational amplifier used as a comparator to sense open or closed path between the conductors (320). The output of the comparator is connected to a relay that opens and closes mimicking the sensor conductor status. The relay in turn switches ON or OFF the vacuum pump to the device (300). The body (301) of the male device (300) includes a handle (309) fixed thereon for holding the device (300). The body (301) also includes a wires conduit (312) and a thumb switch.

Accordingly, the urination episodes are thus monitored and evacuated in real time for in-cognitive users. The vacuum pump (115) is kept ON until the urine bridge between the sensor conductors (320) breaks due to drying of the urine therebetween. This feature also aids in keeping the user dry and preventing collateral infections.

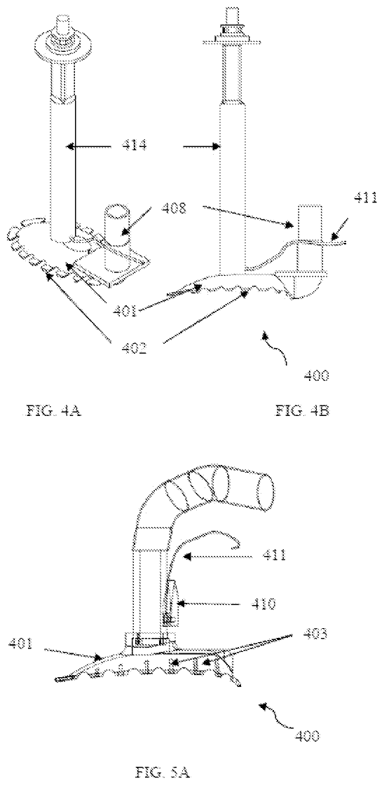

Referring to FIGS. 4A-4C, an urination device (400) for female user (hereinafter referred as (female device (400)) in accordance with an embodiment of the present invention is shown. The female device (400) can be placed over the genitalia, easily and comfortably while the user is lying horizontally in bed. The user is also empowered to independently urinate and have the urine transported to an enclosure.

The female device (400) has a shallow cup shaped body (401). In an embodiment, the profile of the body (401) has an oval shape. The periphery of the body (401) includes a plurality of legs (402) configured thereby forming an undulated shaped contour ensuring air buffer between the pubis of the user and suction inlet of the cup shaped body (401). The leg (402) are configured at the periphery radiating beyond the arches thereby forming a plurality of open channels for extending a suction zone. It is to be noted here that the undulated shaped contour matches with the pubis contour so as to enable snug connection. An inner surface (404) of the body (401) includes a plurality of opposing high velocity air channels (403) configured thereon in a predefined pattern that act as an airport for inrush air. The convexity of the structure is kept nearly flat to minimize volume and maximize the velocity of air flow resulting in efficiency of picking up fluid.

In accordance with an embodiment, the female device (400) includes a handle (408) fixed on an outer surface (406) thereof. The handle (408) is provided with a wire conduit and operating switch platform. The handle (408) is provided for ease of handling and an easy placement of the device (400) on the urethra. The handle (408) also houses an operating switch (410) for the vacuum device with wires (411). The lower middle portion of the body (401) includes a tubular vacuum connector spout (412). The tubular port/vacuum port (412) connects a vacuum tube (414) to a wet vacuum pump device (115). An outlet positioned on the body forms a vacuum tube connector spout (412). The device (400) includes strap loops (420) positioned thereon for placing at least on strap to position the device on the user's body.

In accordance with an embodiment, the oval body (401) of the device (400) has major and minor axis. The opposing high velocity air channels (403) configured on the inner surface (404) of the body (401) facilitate inrush of air to corral the urine as well as raise the surface of the urine closer to the suction inlet of the cup (401). The legs (402) along the perimeter of the device (400) capture stray urine outside the periphery of the device (400). The open ended convex shaped body (401) having perimeter and footprint mirrors the contours of a female genitalia or vulva to enable the device (400) to achieve a snug fit without impairing the flow of vapor, a mixture of air and urine.

In accordance with an embodiment, the structure of the female devices (400) is semi rigid and with a smooth surface finish to make it comfortable to touch. It is a nearly flat convex structure with the following criteria for proportion and shapes. The convex structure is large enough to be able to cover the female genitalia and ease of placement covering the urethra. It is elliptical in footprint with the major axis that spans a little over the urethra to a distance between the legs. The contacting surface mirrors the vulva contours.

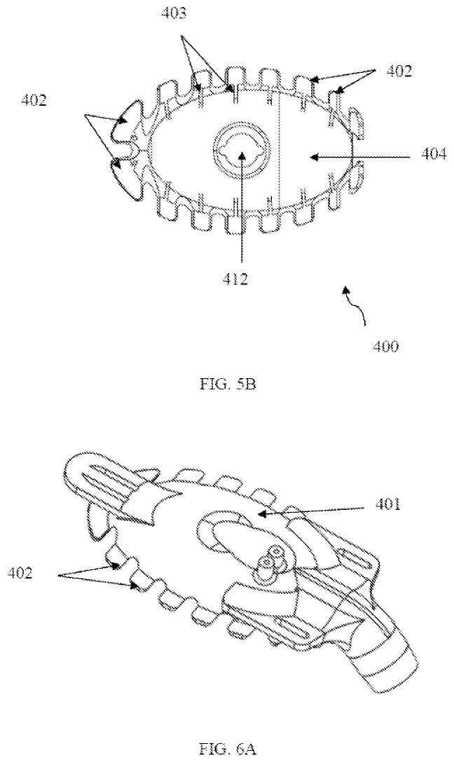

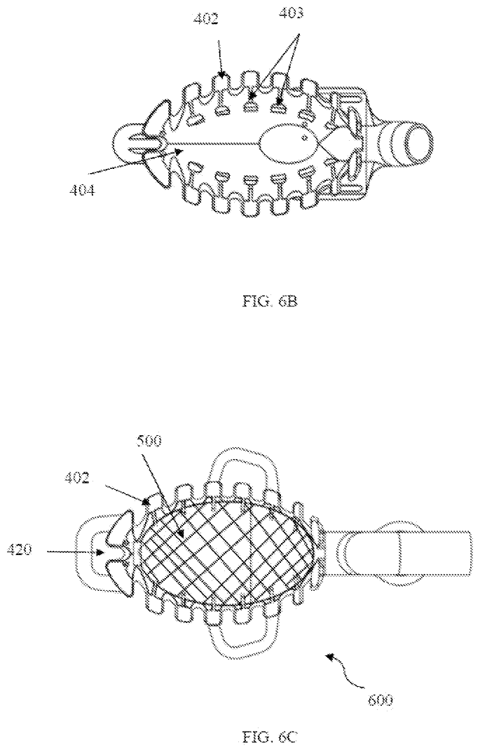

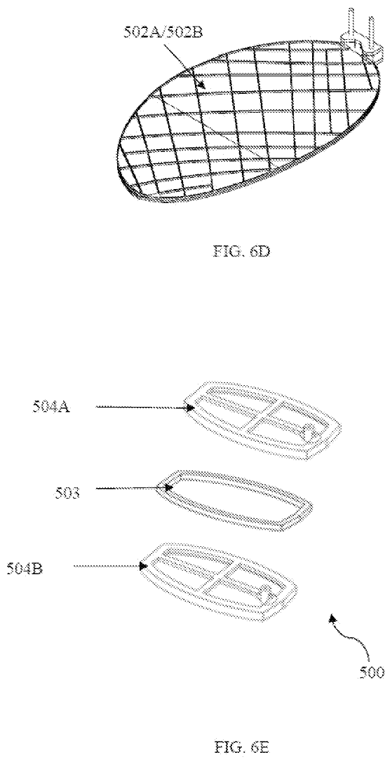

Referring to FIGS. 5A-5B and FIGS. 6C-6E, in alternative embodiment of the present invention, a device (600) for In-Cognitive users is shown. It is recognized that the implementation of sensor for detecting urine for turning ON the vacuum pump (115) when urine is detected and turning OFF the pump (115) upon it being vacuumed OFF, eliminates the need for manual switch even for manual control. This process is noninvasive and ensures the genitals are dry before the vacuum is turned Off. In the device are embedded conductors (please show on the FIGS.) connected with the mesh unit (500). When the mesh is wetted by urine, the conductors are connected together, as the urine being a good conductor that turns ON the vacuum pump until the mesh unit (500) is dried. An isolating circuit isolates the sensor in the device (600) from high voltage. The sensor works on a low DC voltage, preferably, 5V DC. Routinely with certain hours of use the device (600) is sanitized by placing it in the sanitization tray (107) with sanitization fluid.

Referring to FIG. 6A-6C, the device (600) without the handle in position is disclosed. The device (600) has sensors placed therein for detecting urine that serves the purpose to switch the vacuum pump ON and OFF. The device (600) is placed over the pubis using the handle and then urination initiated. The sensor turns ON the vacuum pump (115) until all of it is evacuated and the inside of the device (600) is dry. Once the device is completely dry the pump is turned OFF. The device (600) is then returned to the sanitization tray (107) in a vertical stance. If cleansing by a sanitization fluid is required, the fluid with electrolyte content is manually poured into the tray (107). The vacuum goes through the ON/OFF cycle until the sanitization fluid is completely vacuumed and the device (600) is made ready for the next use. In this embodiment of the present invention, fewer components such as sanitization pump and fewer components in the control box are required.

Referring now to FIG. 6C-6E, a mesh unit (500) of the device (400) is shown. The mesh unit (500) comprises a pair of meshes (502A, 502B) sandwiched between a top cover (504A) and a bottom cover (504B). The mesh unit (500) includes a frame (503). A first mesh (502A) is positioned above the frame (503) and a second mesh (502B) is positioned below the frame (503). The top cover (504A) positions above the first mesh (502A) and the bottom cover (504B) positions below the second mesh (502B) in a snap fit arrangement. The mesh unit (500) enables the urination to take place with a modicum of privacy. Just as male trousers are provided with front opening fly to enable urinating without having to drop the trousers, the mesh cover (5004A, 504B) provides a body cover with a slit with overlapping flaps to allow female users who need to urinate while lying down to insert the urination device for vacuuming the urine. FIG. 6E shows an exploded diagram of the mesh unit (500).

Referring to FIGS. 1 to 6E, in operation, the vacuum pump (115) is powered by turning ON the power by operating the thumb switch (306, 410) that is at the top of the handle of the devices (300, 400). The thumb switch (306, 410) is connected to a low voltage DC source that powers a coil of a relay. The relay has a potential free contact that is used to power the vacuum pump through domestic AC power source. The thumb switch (306, 410) provides inputs to a timer circuit to turn on the vacuum pump (115) that pumps sanitization fluid to a predefined level, preferably from one level to a higher level into a sanitization tray (107). The timer circuit has a first timer that is adjustable to fill the tray (107) to a level that is sufficient to immerse the device (300, 400) during a resting position in the tray before or after use. The timer circuit has a second timer that receives signal to turn ON the vacuum pump after a delay. The vacuum pump (115) turns ON for a fixed adjustable time, enough to vacuum the sanitization fluid in the tray (107) after the device (300, 400) is returned to it. The sanitization fluid is transported into the vacuum canister where it introduces odor killing aromatic. The sanitization fluid also sanitizes the vacuum tube (310, 414) during the transport. The system (100) has the functionality to rinse and disinfect the device (300, 400) electively before, after and between each use. The device (300, 400) as well as the tubes (310, 414) are subject to an automatic cleaning process between uses thereof.

In operation, a flowing fluid in the genital devices (300, 400) is subjected to vacuum which is done progressively. The vacuum pump device (115) creates a suction force that causes an inrush of air in the device (300, 400) that is shaped in accordance with the shape of the genitalia. The device (300, 400) has a handle (304, 408) for ease of handling and placement. The wave contours are distributed along the perimeter of the devices (300, 400). The high velocity air channels (303, 403) and the device volume are designed to accelerate the velocity of the jets of inrushing air and stream close to the surface of the flowing urine.

When the device (300, 400) is placed on the genitalia and urination is initiated, one stream of inrushing air is in the same direction of flow of urine and another in the opposite direction of the urine flow. The contour and high velocity air channels (303, 403) on the periphery are shaped and sized to direct the jets of air stream at the meniscus of the urine stream. The conjunction of the curved meniscus and the high velocity inrush air stream causes a lift. Further, the stream of air that rushes in the opposite direction of the urine flow within the device (300, 400) slows the velocity of the urine stream while the air stream in the same direction accelerates it. Breaking the accelerated urine flow simulates the phenomenon of formation of a tsunami. When flowing water is slowed down a head is created which raises the surface level of the urine stream. A low-pressure area over the raised surface due to vacuum further enhances the surface height of the urine stream like a water spout in nature. This transports the fluid to the mouth of the vacuum port of the device (300, 400). Thereafter, once the fluid contacts the mouth of the vacuum port a combination of wetting of the surface and creation of meniscus seal around the mouth of the tube transports the fluid through the tube to the inlet of a large reservoir. A motor fan combination creates a low pressure in the reservoir which is the force manifest in the vacuum port creating the inrush of air.

The urine transported to the large volume reservoir causes the stream to lose its kinetic energy which triggers the phenomenon of rain inside the reservoir. The influence of gravity on the urine when the velocity of air is reduced causes the fluid to be separated from the air. The urine forms a pool at the bottom of the reservoir and the air continues at lower velocity to the fan blades being spun by the motor of the vacuum device (115). The air is propelled past the fan blades into the ambient through the exhaust port. This air can be bubbled through a water bath for further isolating any urine vapour from the ambient.

The device (600) has sensors placed therein for detecting urine that serves the purpose to switch the vacuum pump ON and OFF. The device (600) is placed over the pubis using the handle and then urination initiated. The sensor turns ON the vacuum pump until all of it is evacuated and the inside of the device (600) is dry. Once the device (600) is completely dry the pump is turned OFF. The device (600) is then returned to the sanitization tray (107) in a vertical stance. If cleansing by a sanitization fluid is required, the fluid with electrolyte content is manually poured into the tray (107). The vacuum goes through the ON/OFF cycle until the sanitization fluid is completely vacuumed and the device is made ready for the next use. In this embodiment of the present invention, fewer components such as sanitization pump and fewer components in the control box are required. The device also can be made by the materials that can be disposed after use.

There are instances that the user urine may eject as dribble and in spurts. In such instances the urine may find a deep path in the female genital that may evade contact with the inrush jet streams. To capture urine which dribbles through the groove, the device (400) is provided with a pocket that is slightly disposed lower between the legs. This profile enables urine flowing under gravity to be also captured by a combination of suction and gravity. Misplacement of the device (300, 400) on the genitalia may cause some urine to be outside the device's cover. The inrush streams of air are ensured a directed high velocity air through spider like leg (402) extensions built into the periphery that captures this and is sucked into the device (300, 400) minimizing the leakage of urine if any.

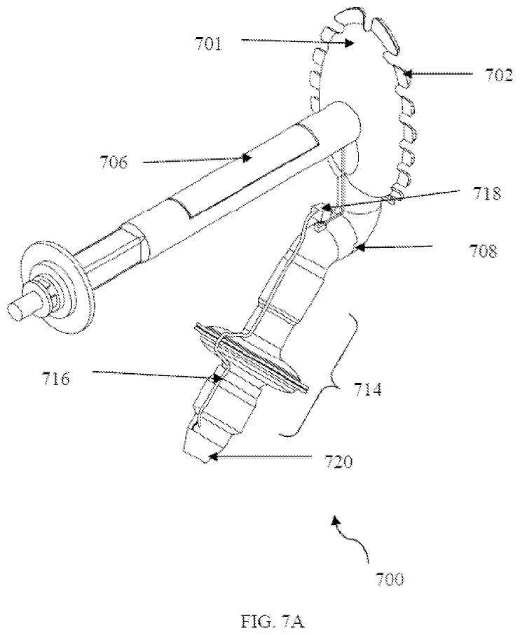

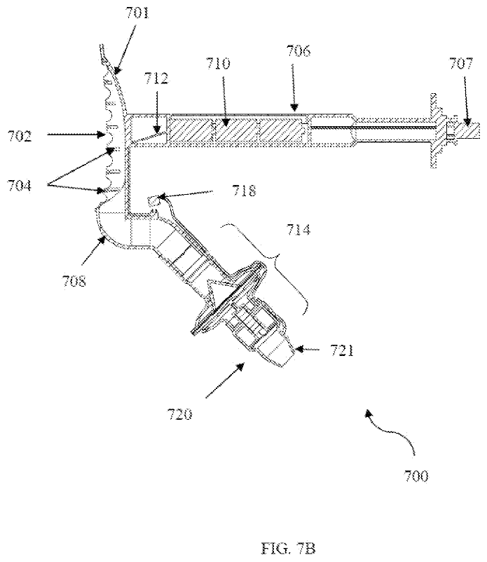

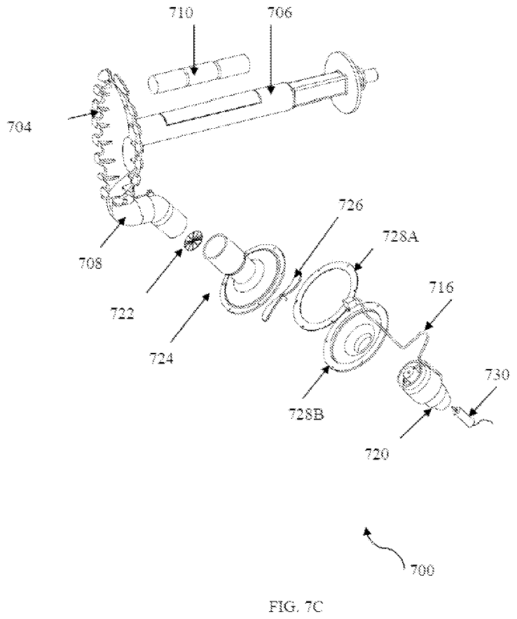

Referring to FIG. 7A-7C, an urination device (700) adapted for female users for urinating in standing position in accordance with an alternative embodiment of the present invention is shown. In this embodiment, the device (700) is a battery powered portable device (700). The device (700) includes a hollow gripping handle (706). The handle (706) houses a power supply unit such as batteries (710). A thumb switch (707) is positioned on the handle (706).

In accordance with this embodiment, the device (700) includes an outlet (708) at one end portion thereof. The outlet (708) is fitted to a suction pipe (709) through an interference fit. The suction pipe (709) includes a vacuum pump assembly (714) placed therein. The vacuum pump assembly (714) receives power supply from the power unit (710) placed in the handle (706). A first electric wire (712) having a plug (718) at end portion thereof is plugged with a second electric wire (716) of the vacuum assembly (714) to complete the circuit.

In accordance with this embodiment, the vacuum assembly (714) is built in two parts to enable insertion of a motor (730) and a propeller (726). An inlet portion of the vacuum assembly (714) has a mesh diaphragm nebulizer (722) to split the urine into droplets that have greater drag and hence easier to vacuum. The mesh diaphragm nebulizer (722) is followed by a cone separator (723) attached to shell walls (724) through struts. The cone separator (723) is configured to split the urine droplets and channelize (728A and 728B) it around the motor (730) positioned at an outlet portion of the vacuum assembly (714). The propeller blades (726) are shaped so that they generate vacuum on the extremities and approximately ⅔^{rd} portion of the propeller (726). The ⅓^{rd} portion of the blades (726) near the axis generate pressure proportioned to channel the sucked urine aerosol vapor around the motor (730) without significant reduction of the vacuum.

The motor (730) is positioned in a housing (720) that forms a cavity between the motor's outer shell and the housing inner shell surface. The cavity forms the shape of an annular ring. When the motor (730) is energized it sucks the urine vapor and channelizes the urine vapor through the annular cavity to exit on the opening of the vacuum assembly (714) away from the device (700). The outlet part opening (721) is tapered to integrate and converge the exiting vapor into a single stream for splash proof disposal. The exiting vapor is directed into any external urinal bowl for disposal.

The motor (730) is connected to the power unit (710) through the plug socket (718) arrangement through the switch (718) mounted on the outlet (708). The power generated through the power unit (710) is conveyed to the motor (730) through a thumb operated switch (707) via ribbon wires (712). The mating of outlet (708) to vacuum assembly (714) is through conducting gaskets (728A, 728B) that is connected to the housing (720) of the motor (730).

In accordance with an embodiment of the present invention, it is understood here that the body (701) of the device (700) contacting the area around the vagina/organ has a undulated profile (702). An inner surface of the body (701) of the device (700) includes a plurality of opposing high velocity air channels (704) configured in a predefined location. The high velocity air channels (704) facilitate flowing and suction of the urine in influence of the vacuum created by the vacuum assembly (714). The urine passed out from the outlet (721) of the vacuum assembly (714) is contained in a sealed enclosure and can be easily transferred to another container that is suitable for the disposal of urine. The sealed enclosure protects the ambient from odor and having sufficient storage volume, does not need to be emptied frequently. It is to be noted here that the device (700) can be made of disposable materials such as PET, Plant Fibre and the like that can be disposed of after use.

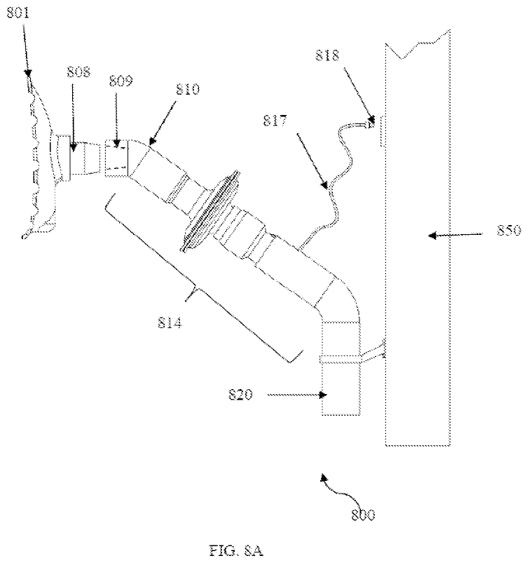

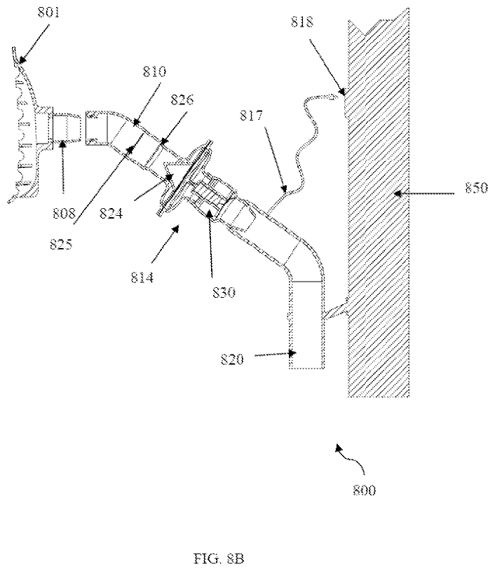

Referring to FIGS. 8A and 8B, a device (800) adapted for female users for urinating in standing position in public urinary in accordance with an alternative embodiment of the present invention is shown. In this embodiment, female users are enabled to use a device (800) for urinating in standing up position. The body of the device (801) along with the outlet (808) is a single mono cog construction that is made up of disposable material to cover the female genital. The body of the device (801) along with the outlet (808) is press fitted into the first section of the vacuum tube assembly (814) having a limit switch (809) positioned therein. The vacuum tube assembly (814) is made of non-disposable material such as PET which is affixed to a structure in the toilet. In accordance with this one embodiment, the disposable material is selected from PET and the like. An outlet (820) of the vacuum tube assembly (814) is shaped for insertion into a member opening that is affixed to a wall (850).

The vacuum tube assembly (814) comprises a cascaded sequence of a suction pipe (810) having a suction end and an exhaust end. The suction pipe (810) has a first section and a second section. The first section having a switch embedded in the suction end of the tube one. A mesh diaphragm nebulizer (825), a cone separator (824), a propeller (826) shaped to provide pressure along a limited zone around the axis and vacuum along the major portion away from the pressure zone extending to the tip of the propeller.

The second section having an inner shell to contain the motor (830), the motor (830) powered through a wire (817) leading out of the co-axial shells to a power source through a plug (818), an outer shell to provide transport corridor between the inner shell and the outer shell and to integrate the vapor into a coalesced unified stream by tapering the shell, a wire (817) that bridges the switch (818) in section one to the motor (830); and an outlet (820) defined at the end portion of the second section.

The first section includes a mesh diaphragm nebulizer (825), a nose cone (824) and a motor propeller unit (830) for suction of the urine. The vacuum tube assembly (814) has an outlet (820) that opens into a drain where the urine is disposed of. The user has to pull out the device (800) from the vacuum tube assembly (814) after use and disposes it off into a hygienic bin. It is to be noted here that the connection is established, and vacuum pump assembly starts to function when the device body (801 and 808) is fixed in the suction pipe (810).

The embodiments of the invention shown and discussed herein are merely illustrative of modes of application of the present invention. Reference to details in this discussion is not intended to limit the scope of the claims to these details, or to the figures used to illustrate the invention.

It is understood that various omission and substitutions of equivalents are contemplated as circumstance may suggest or render expedient, but such are intended to cover the application or implementation without departing from the scope of the present invention.

1.-15. (canceled)

16. A system for urination device, the system comprising:
an enclosure having a sanitization fluid container and an overflow container placed therein;
a sanitization tray placed in the enclosure, the sanitization tray configured to facilitate placement of an urination device therein;
a control circuit for filling and vacuuming the sanitization tray for sanitizing the urination device;
a vacuum pump connected with the control circuit for vacuuming the urine output of the user through the urination device;
a thumb switch configured on a handle of the urination device, the thumb switch is configured to turn ON/OFF the vacuum pump; and
a timer circuit configured to receive the thumb switch input to turn ON the vacuum pump for pumping sanitization fluid into a sanitization tray to a predetermined level.

17. The system according to claim 16, wherein a fluid pump is provided to pump the sanitization fluid into the sanitization tray.

18. The system according to claim 16, wherein the sanitization tray has a drain port and an overflow port.

19. The system according to claim 16, wherein the thumb switch is connected to a low voltage DC source that powers a coil of a relay.

20. A urination device comprising:
a body with a plurality of arches on an outer periphery thereof;
a plurality of legs configured at the periphery radiating beyond the arches thereby forming a plurality of open channels for extending a suction zone;
a plurality of opposing high velocity air channels configured on an inner surface of the body in a predefined pattern that act as an air port for inrush air, the channel walls ensuring an air buffer between the organ on which the device is placed and the suction end of the suction spout;
an outlet positioned on the body forming a vacuum tube connector spout; and
a tube connected to the vacuum tube connector spout at first end of the tube and a second end of the tube connected to a wet vacuum pump device.

21. The urination device according to claim 20, wherein the body of the device has a conical shape or a shallow cup shape.

22. The urination device according to claim 20, wherein the body of the device includes a mesh unit.

23. The urination device according to claim 22, wherein the mesh unit comprises a pair of conducting mesh separated by a fixed distance arranged to cover the open surface of the device that are part of twin elements of sensors for detecting the presence of urine when connected to a commensurate circuit, the pair of mesh being connected to conductors that are inserted through the cup surface to be available for connecting the sensors to a control circuit.

24. The urination device according to claim 22, wherein the body of the device has an inner surface that includes at least one sensor for detection of the urine received therein.

25. The urination device according to claim 23, wherein the body of the device has an inner surface that includes at least one sensor for detection of the urine received therein.

26. The urination device according to claim 20, wherein the body includes a plurality of strap loops for placement on the body of the user.

27. A urination device comprising:
a shallow cup shaped body with a plurality of arches on an outer periphery thereof;
a plurality of legs configured at the periphery radiating out of the ends of arches thereby forming a plurality of open channels for extending a suction zone;
a plurality of opposing high velocity air channels configured on an inner surface of the body in a predefined pattern that act as an air ports for inrush air, the channel walls ensuring an air buffer between the organ on which the device is placed and the suction end of the suction spout;
an outlet positioned at a predefined portion of the body that acts as a vacuum tube connector spout;
a handle having a power unit placed therein, the power unit is configured to supply power through a first wire having a plug;
a suction pipe connected to the vacuum tube connector spout at first end of the suction pipe and a second end of the suction pipe acts as an outlet; and
a vacuum pump assembly positioned at an outlet portion the suction pipe, the vacuum pump assembly is powered from a motor that receives power from the power unit.

28. The urination device according to claim 27, wherein the vacuum tube assembly comprises in the following cascaded sequence:
a suction pipe having a suction end and an exhaust end, the pipe having a first section and a second section,
the first section having
a switch embedded in the suction end of the suction pipe,
a mesh diaphragm nebulizer,
a cone separator configured to split the urine droplets and channelize around the motor,
a propeller shaped to provide pressure along a limited zone around the axis and vacuum along the major portion away from the pressure zone extending to the tip of the propeller,
the second section having
an inner shell to contain the motor, the motor powered through a wire leading out of the co-axial shells to a power source through a plug,
an outer shell to provide transport corridor between the inner shell and the outer shell and to integrate the vapor into a coalesced unified stream by tapering the shell,
a wire that bridges the switch in first section one to the motor; and
an outlet defined at the end portion of the second section.

29. The urination device according to claim 27, wherein the motor is positioned in a housing that forms a cavity between the motor's outer shell and the housing inner shell surface thereby defining an annular ring.

30. The urination device according to claim 28, wherein the motor is positioned in a housing that forms a cavity between the motor outer shell and the housing inner shell surface thereby defining an annular ring.

31. The urination device according to claim 28, wherein the urination device body along with the outlet is made of disposable material and the outlet is press fitted into the first section of the vacuum tube assembly having a limit switch positioned therein.

32. The urination device according to claim 27, wherein the device is made of disposable materials such as PET, Plant Fiber and the like.

33. The urination device according to claim 28, wherein the device is made of disposable materials such as PET, Plant Fiber and the like.
